(19) United States
(12) Patent Application Publication
(10) Pub. No.: US 2022/0202663 A1
(43) Pub. Date: Jun. 30, 2022

(54) SUBSTANTIALLY SILICONE-FREE GELLED COMPOSITIONS

(71) Applicant: CALUMET SPECIALTY PRODUCTS PARTNERS, L.P., Indianapolis, IN (US)

(72) Inventors: Craig QUEEN, Belmont, NC (US); Angela VIERTEL, Parker, PA (US); Mark A. VOELKER, Butler, PA (US)

(21) Appl. No.: 17/609,119

(22) PCT Filed: May 4, 2020
(86) PCT No.: PCT/US2020/031296

§ 371 Date: Nov. 5, 2021

Related U.S. Application Data
(60) Provisional application No. 62/843,696, filed on May 6, 2019.

(51) Int. Cl. A61K 8/04; A61K 8/81; A61Q 19/00; A61Q 5/00

(52) U.S. Cl. CPC A61K 8/042; A61K 8/8117; A61K 2800/522; A61Q 5/00; A61K 2800/34; A61Q 19/00

(57) ABSTRACT

The present disclosure relates to substantially silicone-free gelled compositions.

### CROSS-REFERENCE TO RELATED APPLICATION

This application claims priority to U.S. Provisional Patent Application No. 62/843,696, filed May 6, 2019, the entire disclosure of which is incorporated herein by reference.

### BACKGROUND AND SUMMARY OF THE INVENTION

Silicones are important ingredients that are used in numerous applications across many industries. For instance, silicones provide the necessary functionality and performance in gelled formulations for many personal care applications such as haircare and skin care.

However, the marketplace is suffering from a current shortage of silicones. This scarcity of silicones, coupled with possible health and environmental concerns, has created the need for replacement technologies that do not require silicones to be present. For example, compositions that are substantially free of silicones, or even completely free of silicones, for personal care applications are highly desirable.

Therefore, there exists a need for new compositions and methods that provide gel formulations in which the compositions are substantially free of silicones. Accordingly, the present disclosure provides gelled compositions and methods of making and using the gelled compositions, which exhibit desirable properties and provide related advantages compared to known compositions in the marketplace.

The present disclosure provides gelled composition comprising i) an aliphatic solvent, ii) one or more copolymers, and iii) an antioxidant. The disclosure also provides methods of making the gelled compositions and methods of using the same.

The gelled compositions and methods involving the gelled compositions according to the present disclosure provide several advantages compared to other compositions and methods known in the art. First, the gelled compositions provide the ability to achieve various properties that are desirable in the personal care arena, including viscosity, refractive index, hydrophobicity, volatility, lubricity, sheen/gloss, and others. Second, the gelled compositions allow for the replacement of substantially all silicones in the final compositions, which provides economical, health, and environmental advantages. Third, the gelled compositions match the desirable properties of silicone-containing products, with or without a thickener, but by using ingredients that do not require silicone. Surprisingly, the described gelled compositions utilize an aliphatic solvent as a replacement for silicones yet maintain desirable properties of personal care applications utilizing the gelled compositions.

The following numbered embodiments are contemplated and are non-limiting:
1. A gelled composition comprising i) an aliphatic solvent, ii) one or more copolymers, and iii) an antioxidant.
2. The gelled composition of clause 1, wherein the aliphatic solvent is present in the gelled composition at a concentration of about 80% to about 99.9%.
3. The gelled composition of clause 1 or clause 2, wherein the aliphatic solvent is an isoparaffin.
4. The gelled composition of clause 3, wherein the isoparaffin is present in the gelled composition at a concentration of about 80% to about 99.9%.
5. The gelled composition of any of clauses 1 to 4, wherein the aliphatic solvent is selected from the group consisting of a C7-8 isoparaffin, a C8-9 isoparaffin, a C10-11 isoparaffin, a C11-12 isoparaffin, a C11-13 isoparaffin, a C13-14 isoparaffin, and any combination thereof.
6. The gelled composition of any of clauses 1 to 4, wherein the aliphatic solvent is an

Isopar solvent or a compositional equivalent.
7. The gelled composition of any of clauses 1 to 4, wherein the aliphatic solvent is an Isopar solvent.
8. The gelled composition of clause 7, wherein the Isopar solvent is selected from the group consisting of Isopar M, Isopar C, Isopar E, Isopar G, Isopar H, Isopar L, Isopar V, and any combination thereof.
9. The gelled composition of clause 7, wherein the Isopar solvent is Isopar M.
10. The gelled composition of clause 9, wherein the Isopar M is present in the gelled composition at a concentration of about 80% to about 99.9%.
11. The gelled composition of any of clauses 1 to 10, wherein the copolymer is one or more styrenic block copolymers.
12. The gelled composition of any of clauses 1 to 11, wherein the copolymer is present in the gelled composition at a concentration of about 0.1% to about 20%.
13. The gelled composition of any of clauses 1 to 12, wherein the gelled composition comprises Kraton G 1702.
14. The gelled composition of clause 13, wherein the Kraton G 1702 is present in the gelled composition at a concentration of about 0.1% to about 10%.
15. The gelled composition of any of clauses 1 to 14, wherein the gelled composition comprises a first copolymer, wherein the first copolymer is Kraton G 1702, and optionally comprises a second copolymer.
16. The gelled composition of clause 15, wherein the Kraton G 1702 is present in the gelled composition at a concentration of about 0.1% to about 10%.
17. The gelled composition of any of clauses 1 to 12, wherein the copolymer comprises Kraton G 1650.
18. The gelled composition of clause 17, wherein the Kraton G 1650 is present in the gelled composition at a concentration of about 0.1% to about 10%.
19. The gelled composition of any of clauses 1 to 14, wherein the gelled composition comprises a first copolymer, wherein the first copolymer is Kraton G 1650, and optionally comprises a second copolymer.
20. The gelled composition of clause 19, wherein the Kraton G 1650 is present in the gelled composition at a concentration of about 0.1% to about 10%.
21. The gelled composition of any of clauses 1 to 20, wherein the gelled composition comprises two copolymers.
22. The gelled composition of clause 21, wherein one copolymer is Kraton G 1702 and the second copolymer is Kraton G 1650.
23. The gelled composition of clause 22, wherein the Kraton G 1702 is present in the gelled composition at a concentration of about 0.1% to about 10% and the Kraton G 1650 is present in the gelled composition at a concentration of about 0.1% to about 10%.
24. The gelled composition of clause 22, wherein the Kraton G 1702 is present in the gelled composition at a concentration of about 4% to about 10% and the Kraton G 1650 is present in the gelled composition at a concentration of about 0.1% to about 1.0%.
25. The gelled composition of any of clauses 1 to 24, wherein the antioxidant is Tinogard®.
26. The gelled composition of clause 25, wherein the Tinogard® is present in the gelled composition at a concentration of about 0.01% to about 0.1%.
27. The gelled composition of clause 25,wherein the Tinogard® is present in the gelled composition at a concentration of about 0.01% to about 0.05%.
28. The gelled composition of any of clauses 1 to 27, wherein the gelled composition is substantially free of a silicone.
29. The gelled composition of any of clauses 1 to 27, wherein the gelled composition does not comprise a silicone.
30. The gelled composition of any of clauses 1 to 29, wherein the gelled composition is substantially free of a thickener.
31. The gelled composition of any of clauses 1 to 29, wherein the gelled composition does not comprise a thickener.
32. The gelled composition of any of clauses 1 to 31, wherein the gelled composition is not a lotion.
33. The gelled composition of any of clauses 1 to 32, wherein the gelled composition has a viscosity from about 5,000 cps to about 50,000 cps.
34. The gelled composition of any of clauses 1 to 32, wherein the gelled composition has a viscosity from about 5,000 cps to about 10,000 cps, or wherein the gelled composition has a viscosity from about 10,000 cps to about 20,000 cps.
35. The gelled composition of any of clauses 1 to 32, wherein the gelled composition has a viscosity from about 10,000 cps to about 15,000 cps.
36. The gelled composition of any of clauses 1 to 32, wherein the gelled composition has a viscosity from about 15,000 cps to about 20,000 cps.
37. The gelled composition of any of clauses 1 to 32, wherein the gelled composition has a viscosity from about 20,000 cps to about 50,000 cps.
38. The gelled composition of any of clauses 1 to 32, wherein the gelled composition has a viscosity from about 30,000 cps to about 50,000 cps.
39. The gelled composition of any of clauses 1 to 32, wherein the gelled composition has a viscosity from about 40,000 cps to about 50,000 cps.
40. The gelled composition of any of clauses 1 to 32, wherein the gelled composition has a viscosity of about 5,000 cps.
41. The gelled composition of any of clauses 1 to 32, wherein the gelled composition has a viscosity of about 10,000 cps.
42. The gelled composition of any of clauses 1 to 32, wherein the gelled composition has a viscosity of about 15,000 cps.
43. The gelled composition of any of clauses 1 to 32, wherein the gelled composition has a viscosity of about 20,000 cps.
44. The gelled composition of any of clauses 1 to 32, wherein the gelled composition has a viscosity of about 25,000 cps.
45. The gelled composition of any of clauses 1 to 32, wherein the gelled composition has a viscosity of about 30,000 cps.
46. The gelled composition of any of clauses 1 to 32, wherein the gelled composition has a viscosity of about 35,000 cps.
47. The gelled composition of any of clauses 1 to 32, wherein the gelled composition has a viscosity of about 40,000 cps.
48. The gelled composition of any of clauses 1 to 32, wherein the gelled composition has a viscosity of about 45,000 cps.
49. The gelled composition of any of clauses 1 to 32, wherein the gelled composition has a viscosity of about 50,000 cps.
50. A composition comprising the gelled composition of clause 1 and an oil.
51. The composition of clause 50, wherein the gelled composition is the gelled composition of any of clauses 1 to 49.
52. The composition of clause 50 or clause 51, wherein the oil is sunflower oil.
53. The composition of clause 50 or clause 51, wherein the oil is selected from the group consisting of apricot oil, avocado oil, borage oil, castor oil, coconut oil, evening primrose oil, gold of pleasure oil, grape seed oil, hazelnut oil, jojoba oil, macadamia oil, passionflower oil, peach kernel oil, rice bran oil, safflower oil, sesame oil, shea oleine oil, sweet almond oil, almond oil, wheat germ oil, sunflower oil, and any combination thereof.
54. The composition of any of clauses 50 to 53, wherein the composition is a hair care composition.
55. The composition of any of clauses 50 to 53, wherein the composition is a skin care composition.
56. The composition of any of clauses 50 to 53, wherein the composition is a personal care composition.
57. A gelled composition comprising i) an aliphatic solvent, ii) a silicone, iii) one or more copolymers, and iv) an antioxidant.
58. The gelled composition of clause 57, wherein the aliphatic solvent is present in the gelled composition at a concentration of about 1% to about 50%.
59. The gelled composition of clause 57 or clause 58, wherein the aliphatic solvent is an isoparaffin.
60. The gelled composition of clause 59, wherein the isoparaffin is present in the gelled composition at a concentration of about 1% to about 50%.
61. The gelled composition of any of clauses 57 to 60, wherein the aliphatic solvent is selected from the group consisting of a C7-8 isoparaffin, a C8-9 isoparaffin, a C10-11 isoparaffin, a C11-12 isoparaffin, a C11-13 isoparaffin, a C13-14 isoparaffin, and any combination thereof.
62. The gelled composition of any of clauses 57 to 60, wherein the aliphatic solvent is an Isopar solvent or a compositional equivalent.
63. The gelled composition of any of clauses 57 to 60, wherein the aliphatic solvent is an Isopar solvent.
64. The gelled composition of clause 63, wherein the Isopar solvent is selected from the group consisting of Isopar M, Isopar C, Isopar E, Isopar G, Isopar H, Isopar L, Isopar V, and any combination thereof.
65. The gelled composition of clause 63, wherein the Isopar solvent is Isopar M.
66. The gelled composition of clause 65, wherein the Isopar M is present in the gelled composition at a concentration of about 1% to about 50%.
67. The gelled composition of any of clauses 57 to 66, wherein the silicone is present in the gelled composition at a concentration of about 1% to about 50%.
68. The gelled composition of any of clauses 57 to 67, wherein the silicone is selected from the group consisting of cyclotetrasiloxane, trimethicone, dimethiconol, cyclomethicone, dimethicone, methicone, phenyl trimethicone, and any combination thereof.
69. The gelled composition of any of clauses 57 to 68, wherein the copolymer is one or more styrenic block copolymers.
70. The gelled composition of any of clauses 57 to 69, wherein the copolymer is present in the gelled composition at a concentration of about 0.1% to about 20%.
71. The gelled composition of any of clauses 57 to 70, wherein the gelled composition comprises Kraton G 1702.
72. The gelled composition of clause 71, wherein the Kraton G 1702 is present in the gelled composition at a concentration of about 0.1% to about 10%.
73. The gelled composition of any of clauses 57 to 70, wherein the gelled composition comprises a first copolymer, wherein the first copolymer is Kraton G 1702, and optionally comprises a second copolymer.
74. The gelled composition of clause 73, wherein the Kraton G 1702 is present in the gelled composition at a concentration of about 0.1% to about 10%.
75. The gelled composition of any of clauses 57 to 74, wherein the copolymer comprises Kraton G 1650.
76. The gelled composition of clause 75, wherein the Kraton G 1650 is present in the gelled composition at a concentration of about 0.1% to about 10%.
77. The gelled composition of any of clauses 57 to 76, wherein the gelled composition comprises a first copolymer, wherein the first copolymer is Kraton G 1650, and optionally comprises a second copolymer.
78. The gelled composition of clause 77, wherein the Kraton G 1650 is present in the gelled composition at a concentration of about 0.1% to about 10%.
79. The gelled composition of any of clauses 57 to 78, wherein the gelled composition comprises two copolymers.
80. The gelled composition of clause 79, wherein one copolymer is Kraton G 1702 and the second copolymer is Kraton G 1650.
81. The gelled composition of clause 80, wherein the Kraton G 1702 is present in the gelled composition at a concentration of about 0.1% to about 10% and the Kraton G 1650 is present in the gelled composition at a concentration of about 0.1% to about 10%.
82. The gelled composition of clause 80, wherein the Kraton G 1702 is present in the gelled composition at a concentration of about 4% to about 10% and the Kraton G 1650 is present in the gelled composition at a concentration of about 0.1% to about 1.0%.
83. The gelled composition of any of clauses 57 to 82, wherein the antioxidant is Tinogard®.
84. The gelled composition of clause 83, wherein the Tinogard® is present in the gelled composition at a concentration of about 0.01% to about 0.1%.
85. The gelled composition of clause 83, wherein the Tinogard® is present in the gelled composition at a concentration of about 0.01% to about 0.05%.
86. The gelled composition of any of clauses 57 to 85, wherein the gelled composition is substantially free of a silicone.
87. The gelled composition of any of clauses 57 to 85, wherein the gelled composition does not comprise a silicone.
88. The gelled composition of any of clauses 57 to 87, wherein the gelled composition is substantially free of a thickener.
89. The gelled composition of any of clauses 57 to 87, wherein the gelled composition does not comprise a thickener.
90. The gelled composition of any of clauses 57 to 89, wherein the gelled composition is not a lotion.
91. The gelled composition of any of clauses 57 to 90, wherein the gelled composition has a viscosity from about 5,000 cps to about 50,000 cps.
92. The gelled composition of any of clauses 57 to 90, wherein the gelled composition has a viscosity from about 5,000 cps to about 10,000 cps.
93. The gelled composition of any of clauses 57 to 90, wherein the gelled composition has a viscosity from about 10,000 cps to about 20,000 cps.
94. The gelled composition of any of clauses 57 to 90, wherein the gelled composition has a viscosity from about 10,000 cps to about 15,000 cps.
95. The gelled composition of any of clauses 57 to 90, wherein the gelled composition has a viscosity from about 15,000 cps to about 20,000 cps.
96. The gelled composition of any of clauses 57 to 90, wherein the gelled composition has a viscosity from about 20,000 cps to about 50,000 cps.
97. The gelled composition of any of clauses 57 to 90, wherein the gelled composition has a viscosity from about 30,000 cps to about 50,000 cps.
98. The gelled composition of any of clauses 57 to 90, wherein the gelled composition has a viscosity from about 40,000 cps to about 50,000 cps.
99. The gelled composition of any of clauses 57 to 90, wherein the gelled composition has a viscosity of about 5,000 cps.
100. The gelled composition of any of clauses 57 to 90, wherein the gelled composition has a viscosity of about 10,000 cps.
101. The gelled composition of any of clauses 57 to 90, wherein the gelled composition has a viscosity of about 15,000 cps.
102. The gelled composition of any of clauses 57 to 90, wherein the gelled composition has a viscosity of about 20,000 cps.
103. The gelled composition of any of clauses 57 to 90, wherein the gelled composition has a viscosity of about 25,000 cps.
104. The gelled composition of any of clauses 57 to 90, wherein the gelled composition has a viscosity of about 30,000 cps.
105. The gelled composition of any of clauses 57 to 90, wherein the gelled composition has a viscosity of about 35,000 cps.
106. The gelled composition of any of clauses 57 to 90, wherein the gelled composition has a viscosity of about 40,000 cps.
107. The gelled composition of any of clauses 57 to 90, wherein the gelled composition has a viscosity of about 45,000 cps.
108. The gelled composition of any of clauses 57 to 90, wherein the gelled composition has a viscosity of about 50,000 cps.
109. A composition comprising the gelled composition of clause 57 and an oil.
110. The composition of clause 109, wherein the gelled composition is the gelled composition of any of clauses 57 to 107.
111. The composition of clause 109 or clause 110, wherein the oil is selected from the group consisting of apricot oil, avocado oil, borage oil, castor oil, coconut oil, evening primrose oil, gold of pleasure oil, grape seed oil, hazelnut oil, jojoba oil, macadamia oil, passionflower oil, peach kernel oil, rice bran oil, safflower oil, sesame oil, shea oleine oil, sweet almond oil, almond oil, wheat germ oil, sunflower oil, and any combination thereof.
112. The composition of any of clauses 109 to 111, wherein the oil is sunflower oil.
113. The composition of any of clauses 109 to 112, wherein the composition is a hair care composition.
114. The composition of any of clauses 109 to 112, wherein the composition is a skin care composition.
115. The composition of any of clauses 109 to 112, wherein the composition is a personal care composition.
116. A method of making a gelled composition, said method comprising the steps of a) combining an aliphatic solvent, one or more copolymers, and an antioxidant, and b) mixing the combination to form the gelled composition.
117. The method of clause 116, wherein the gelled composition is the gelled composition of any of clauses 1 to 49.
118. The method of clause 116, wherein the method further comprises the step of combining a silicone to form the gelled composition.
119. The method of clause 118, wherein the gelled composition is the gelled composition of any of clauses 57 to 108.
120. The method of any of clauses 116 to 119, wherein the mixing takes place at a temperature between about 80° F. and about 280° F.
121. The method of any of clauses 116 to 119, wherein the mixing takes place at a temperature between about 100° F. and about 250° F.
122. The method of any of clauses 116 to 119, wherein the mixing takes place at a temperature between about 120° F. and about 220° F.
123. The method of any of clauses 116 to 119, wherein the mixing takes place at a temperature between about 140° F. and about 200° F.
124. The method of any of clauses 116 to 119, wherein the mixing takes place at a temperature between about 150° F. and about 190° F.
125. The method of any of clauses 116 to 119, wherein the mixing takes place at a temperature between about 160° F. and about 180° F.
126. The method of any of clauses 116 to 119, wherein the mixing takes place at a temperature between about 180° F. and about 190° F.

### BRIEF DESCRIPTION OF THE DRAWINGS

FIG. 1 shows the testing setup for evaluation of firmness, stickiness, stringiness, and adhesiveness.

FIG. 2 shows the firmness, stickiness, stringiness, and adhesiveness of Benchmark Composition B2.

FIG. 3 shows the firmness, stickiness, stringiness, and adhesiveness of Prototype Composition P5.

FIG. 4 shows the firmness properties (g) of Benchmark Compositions B1-B3 compared to Prototype Compositions P1-P6.

FIG. 5 shows the stickiness properties (g) of Benchmark Compositions B1-B3 compared to Prototype Compositions P1-P6.

FIG. 6 shows the stringiness properties (mm) of Benchmark Compositions B1-B3 compared to Prototype Compositions P1-P6.

FIG. 7 shows the adhesive work properties (g·mm) of Benchmark Compositions B1-B3 compared to Prototype Compositions P1-P6.

FIG. 8 shows the testing setup for evaluation of lubricity.

FIG. 9 shows the lubricity (friction reduction) of Benchmark Composition B2.

FIG. 10 shows the lubricity (friction reduction) of Prototype Composition P5.

FIG. 11 shows the lubricity (g) of Benchmark Compositions B1-B3 compared to Prototype Compositions P1-P6.

FIG. 12 shows the Refractive Index of Benchmark Compositions B1-B3 compared to Prototype Compositions P1-P6.

FIG. 13 shows summary of product performance of various Prototype Compositions compared to the Benchmark Compositions.

Various embodiments of the invention are described herein as follows. In one embodiment described herein, a gelled composition is provided. The gelled composition comprises i) an aliphatic solvent, ii) one or more copolymers, and iii) an antioxidant.

In another embodiment, another gelled composition is provided. This gelled composition comprises i) an aliphatic solvent, ii) a silicone, iii) one or more copolymers, and iv) an antioxidant.

In yet another embodiment, composition comprising a gelled compositions and an oil is provided.

In another embodiment, a method of making a gelled composition is provided. The method comprises the steps of a) combining an aliphatic solvent, one or more copolymers, and an antioxidant, and b) mixing the combination to form the gelled composition.

In the various embodiments, the gelled composition comprises an aliphatic solvent. As used herein, an aliphatic solvent refers to a solvent that is non-aromatic. In some embodiments, the aliphatic solvent is an isoparaffin. As used herein, an isoparaffin refers to a branched chain hydrocarbon. For instance, isoparaffins may include the Isopar™ fluids, which are described at www.exxonmobilchemical.com/en/˜/media/EB0D6F350FFD4BF782664F57343486E6.ashx.

In the various embodiments, the gelled composition comprises one or more copolymers. In some embodiments, the copolymers can be one or more styrenic block copolymers, which are well known in the art. For instance, copolymers may include the Kraton™ brand of copolymers, which are described at www.kraton.com/.

In the various embodiments, the gelled composition comprises an antioxidant. For instance, an antioxidant may include the Tinogard® brand antioxidants.

In various embodiments, the gelled composition is substantially free of a silicone. In other embodiments, the gelled composition is substantially free of a thickener. As used herein, the term “substantially free” refers to zero or nearly no detectable amount of a material, quantity, or item. For example, the amount can be less than 2 percent, less than 0.5 percent, or less than 0.1 percent of the material, quantity, or item.

Various embodiments of the present disclosure, and combinations thereof, are found in the numbered embodiment list contained herein.

EXAMPLE 1

### Preparation of Gelled Compositions

Gelled compositions of the present disclosure were prepared and evaluated for viscosity and for color. In the instant example, Iospar M was utilized as the exemplary aliphatic solvent. Furthermore, Kraton G 1702 and Kraton G 1650 were utilized as the exemplary copolymers. Tinogard was used as the exemplary antioxidant.

To prepare the gelled compositions of the present disclosure, the Iospar M, Kraton G 1702, Kraton G 1650, and Tinogard were first combined at about 170° F. The combination was then mixed at about 180° F. to about 190° F. for approximately six hours. Viscosity and color of the resultant gelled composition were then evaluated.

Four comparative gelled compositions containing silicone solvents (i.e., Dow Corning 3901 Liquid Satin) were prepared and evaluated in comparison to the gelled compositions of the present disclosure.

To prepare the comparative gelled compositions of the present disclosure, the Iospar M, Kraton G 1702, Kraton G 1650, and Tinogard were first combined at about 180° F. After one hour, the Dow Corning 3901 Liquid Satin was heated to 115° F. and added to the combination. This final combination was then mixed at about 180° F. to about 190° F. for approximately six hours. Viscosity and color of the resultant gelled composition were then evaluated.

### Gelled Composition 1

The ingredients of the gelled composition were as follows:

| | |
|---|---|
| 90.0% | Isopar M |
| 9.68% | Kraton G 1702 |
| 0.3% | Kraton G 1650 |
| 0.02% | Tinogard |

Upon evaluation at 25° C. (T-C, 5 rpm), the viscosity of the gelled composition was 131,200 cps. The saybolt color of the gelled composition was 26.

### Gelled Composition 2

The ingredients of the gelled composition were as follows:

| | |
|---|---|
| 91.9% | Isopar M |
| 7.78% | Kraton G 1702 |
| 0.3% | Kraton G 1650 |
| 0.02% | Tinogard |

Upon evaluation at 25° C. (T-C, 5 rpm), the viscosity of the gelled composition was 36,800 cps. The saybolt color of the gelled composition was 27.

### Gelled Composition 3

The ingredients of the gelled composition were as follows:

| | |
|---|---|
| 92.68% | Isopar M |
| 7.00% | Kraton G 1702 |
| 0.3% | Kraton G 1650 |
| 0.02% | Tinogard |

Upon evaluation at 25° C. (T-C, 5 rpm), the viscosity of the gelled composition was 29,600 cps. The saybolt color of the gelled composition was 27.

### Gelled Composition 4

The ingredients of the gelled composition were as follows:

| | |
|---|---|
| 93.68% | Isopar M |
| 6.00% | Kraton G 1702 |
| 0.3% | Kraton G 1650 |
| 0.02% | Tinogard |

Upon evaluation at 25° C. (T-C, 5 rpm), the viscosity of the gelled composition was 22,400 cps. The saybolt color of the gelled composition was 27.

### Gelled Composition 5

The ingredients of the gelled composition were as follows:

| | |
|---|---|
| 94.53% | Isopar M |
| 5.15% | Kraton G 1702 |
| 0.3% | Kraton G 1650 |
| 0.02% | Tinogard |

Upon evaluation at 25° C. (T-C, 20 rpm), the viscosity of the gelled composition was 5,900 cps. The saybolt color of the gelled composition was 27.

### Gelled Composition 6

The ingredients of the gelled composition were as follows:

| | |
|---|---|
| 94.18% | Isopar M |
| 5.50% | Kraton G 1702 |
| 0.3% | Kraton G 1650 |
| 0.02% | Tinogard |

Upon evaluation at 25° C. (T-C, 10 rpm), the viscosity of the gelled composition was 11,600 cps. The saybolt color of the gelled composition was 27.

### Comparative Gelled Composition A

The ingredients of the comparative gelled composition were as follows:

| | |
|---|---|
| 85.0% | Isopar M |
| 5.0% | Dow Corning 3901 Liquid Satin |
| 9.68% | Kraton G 1702 |
| 0.3% | Kraton G 1650 |
| 0.02% | Tinogard |

Upon evaluation at 25° C. (T-C, 5 rpm), the viscosity of the comparative gelled composition was 70,400 cps. The saybolt color of the comparative gelled composition was very hazy and opaque.

### Comparative Gelled Composition B

The ingredients of the comparative gelled composition were as follows:

| | |
|---|---|
| 80.0% | Isopar M |
| 10.0% | Dow Corning 3901 Liquid Satin |
| 9.68% | Kraton G 1702 |
| 0.3% | Kraton G 1650 |
| 0.02% | Tinogard |

Upon evaluation at 25° C. (T-C, 5 rpm), the viscosity of the comparative gelled composition was 76,400 cps. The saybolt color of the comparative gelled composition was very hazy and opaque.

### Comparative Gelled Composition C

The ingredients of the comparative gelled composition were as follows:

| | |
|---|---|
| 88.68% | Isopar M |
| 5.0% | Dow Corning 3901 Liquid Satin |
| 6.00% | Kraton G 1702 |
| 0.3% | Kraton G 1650 |
| 0.02% | Tinogard |

Upon evaluation at 25° C. (T-C, 5 rpm), the viscosity of the comparative gelled composition was 23,400 cps. The saybolt color of the comparative gelled composition was very hazy and opaque.

### Comparative Gelled Composition D

The ingredients of the comparative gelled composition were as follows:

| | |
|---|---|
| 89.18% | Isopar M |
| 10.0% | Dow Corning 3901 Liquid Satin |
| 5.50% | Kraton G 1702 |
| 0.3% | Kraton G 1650 |
| 0.02% | Tinogard |

Upon evaluation at 25° C. (T-C, 10 rpm), the viscosity of the comparative gelled composition was 12,500 cps. The saybolt color of the comparative gelled composition was very hazy and opaque.

Surprisingly, the addition of the silicone solvent was not as efficient as modification of the copolymers in order to control viscosity of the gelled compositions. In other words, viscosity of the gelled compositions was able to be altered without requiring the addition of silicones to the gelled composition. Furthermore, thickeners were not necessary to formulate the gelled compositions of the present disclosure. In summary, gelled compositions of the present disclosure were able to achieve desirable properties for personal care applications without the requirement of silicones and thickeners but, instead, by incorporating the aliphatic solvent.

### EXAMPLE 2

### Evaluation of Gelled Compositions

Various properties of gelled compositions of the present disclosure were compared to marketed, benchmark compositions. In particular, properties such as firmness, stickiness, stringiness, and adhesiveness were evaluated.

A gelled composition of the present disclosure was combined with an oil or oil-derived component to form a Prototype Composition. In particular, six Prototype Compositions (P1-P6) were formulated as follows using “Gelled Composition 6” as described in Example 1:
Prototype Composition 1 (P1): 90% gelled composition (Isopar M, Kraton G 1702, Kraton G 1650, Tinogard)+10% sunflower oil
Prototype Composition 2 (P2): 80% gelled composition (Isopar M, Kraton G 1702, Kraton G 1650, Tinogard)+20% sunflower oil
Prototype Composition 3 (P3): 70% gelled composition (Isopar M, Kraton G 1702, Kraton G 1650, Tinogard)+30% sunflower oil
Prototype Composition 4 (P4): 60% gelled composition (Isopar M, Kraton G 1702, Kraton G 1650, Tinogard)+40% sunflower oil
Prototype Composition 5 (P5): 50% gelled composition (Isopar M, Kraton G 1702, Kraton G 1650, Tinogard)+50% sunflower oil
Prototype Composition 6 (P6): 50% gelled composition (Isopar M, Kraton G 1702, Kraton G 1650, Tinogard)+50% dodecanol

In comparison to the six Prototype Compositions, three marketed, benchmark compositions (B1-B3) were also evaluated. The properties of the three marketed, benchmark compositions were as follows:
Benchmark Composition 1 (B1): Cyclopentasiloxane, Dimethiconol, Fragrance, Sunflower oil, and Coconut oil
Benchmark Composition 2 (B2): Dimethicone, Cyclopentasiloxane, Water, Fragrance, and Sea buckthorn oil
Benchmark Composition 3 (B3): Cyclopentasiloxane, Isododecane, C12-C15 Alkyl Benzoate, Kukui oil, and Phenyl trimethicone

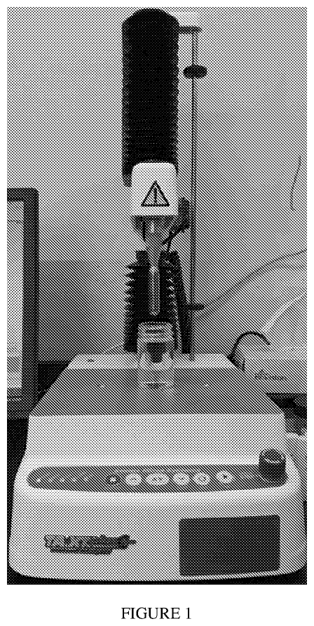

The testing setup for evaluation of firmness, stickiness, stringiness, and adhesiveness is depicted in FIG. 1. Testing parameters were as follows:

| Test mode | Compression | |
|---|---|---|
| Pre-test speed | 3 | mm/s |
| Post-test speed | 5 | mm/s |
| Target mode | Distance | |
| Distance | 10 | mm |
| Trigger type | Auto (Force) | |
| Trigger force | 2 | g |

| | |
|---|---|
| Probe | TA-23 |
| Points/s | 200 |

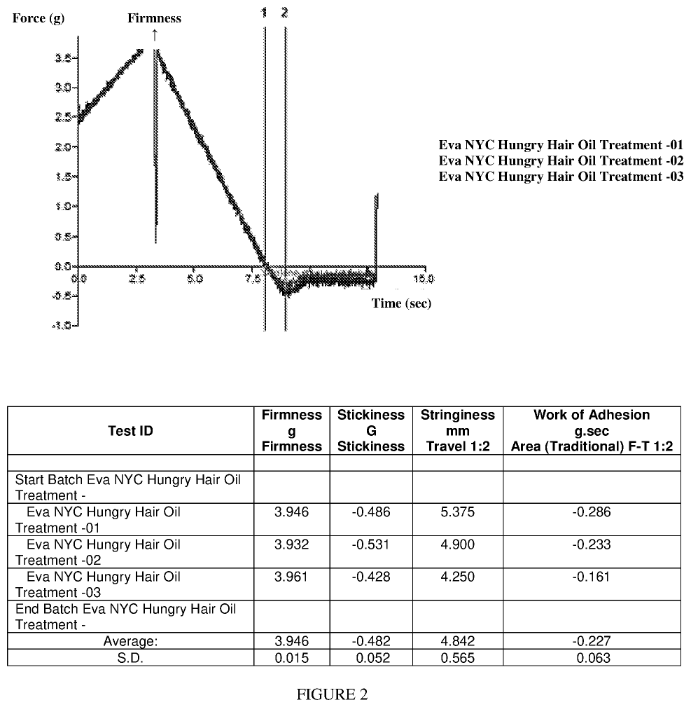

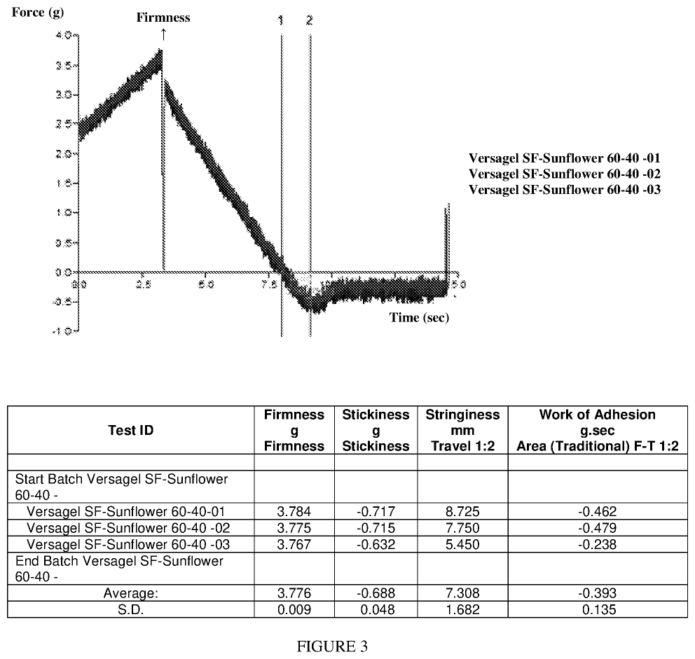

FIG. 2 and FIG. 3 graphically demonstrate the firmness, stickiness, stringiness, and adhesiveness of Benchmark Composition B2 (FIG. 2) compared to Prototype Composition P5 (FIG. 3).

### Firmness

The firmness (e.g., structure or ease of application) of the Benchmark Compositions B1-B3 were compared to Prototype Compositions P1-P6. Firmness was evaluated as the force of probe as it breaks the surface of the sample.

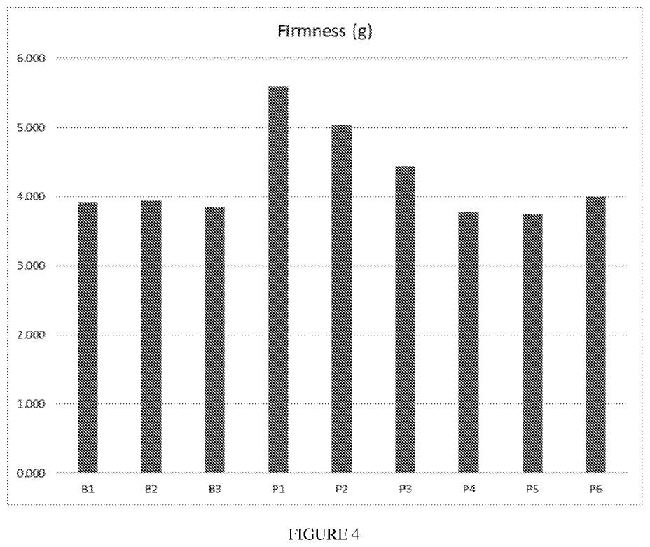

FIG. 4 displays the firmness properties (g) of B1-B3 compared to P1-P6. The three Benchmark Compositions had similar firmness. In comparison, Prototype Compositions P4, P5, and P6 had the most similar firmness values to the Benchmark Compositions.

### Stickiness

The stickiness (e.g., tackiness) of the Benchmark Compositions B1-B3 were compared to Prototype Compositions P1-P6. Stickiness was evaluated as the force exerted by probe as it pulls up from the sample.

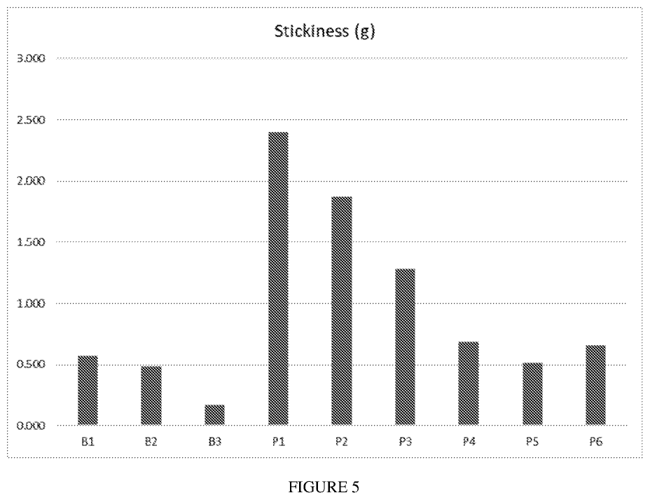

FIG. 5 displays the stickiness properties (g) of B1-B3 compared to P1-P6. The Benchmark Compositions B1 and B2 had similar stickiness. In comparison, Prototype Compositions P4, P5, and P6 had the most similar stickiness values to the Benchmark Compositions B1 and B2.

### Stringiness

The stringiness of the Benchmark Compositions B1-B3 were compared to Prototype Compositions P1-P6. Stringiness was evaluated as the distance over which sample clings to retracting probe.

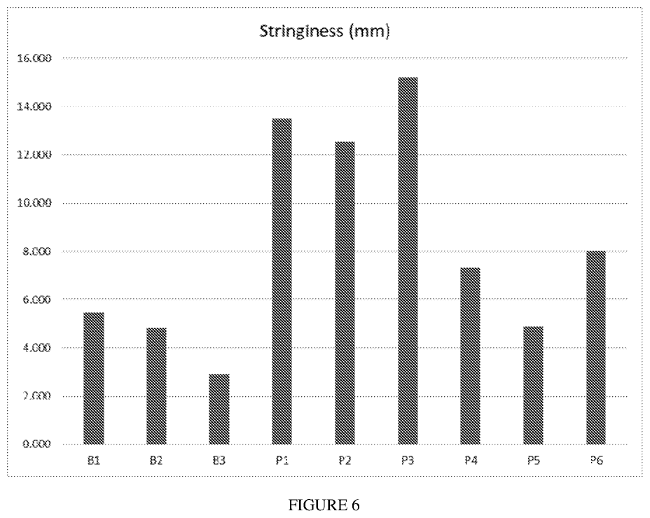

FIG. 6 displays the stringiness properties (mm) of B1-B3 compared to P1-P6. The Benchmark Compositions B1 and B2 had similar stringiness. In comparison, Prototype Compositions P4, P5, and P6 had the most similar stringiness values to the Benchmark Compositions B1 and B2.

### Work of Adhesion

The work of adhesion of the Benchmark Compositions B1-B3 were compared to Prototype Compositions P1-P6. Work of adhesion was evaluated as the work done by the probe to free itself from the sample.

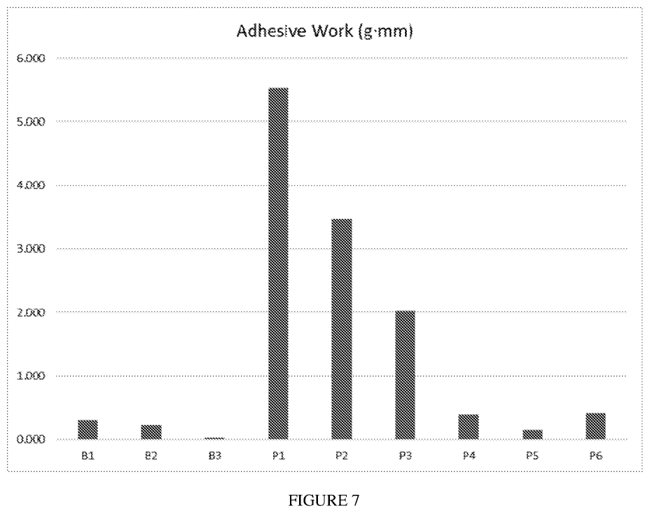

FIG. 7 displays the adhesive work properties (g·mm) of B1-B3 compared to P1-P6. The Benchmark Compositions B1 and B2 had similar adhesive work. In comparison, Prototype Compositions P4, P5, and P6 had the most similar adhesive work values to the Benchmark Compositions B1 and B2.

### EXAMPLE 3

### Lubricity Evaluation of Gelled Compositions

Lubricity properties of gelled compositions of the present disclosure were compared to marketed, benchmark compositions.

A gelled composition of the present disclosure was combined with an oil or oil-derived component to form a Prototype Composition. In particular, six Prototype Compositions (P1-P6) were formulated as follows using “Gelled Composition 6” as described in Example 1:
Prototype Composition 1 (P1): 90% gelled composition (Isopar M, Kraton G 1702, Kraton G 1650, Tinogard)+10% sunflower oil
Prototype Composition 2 (P2): 80% gelled composition (Isopar M, Kraton G 1702, Kraton G 1650, Tinogard)+20% sunflower oil
Prototype Composition 3 (P3): 70% gelled composition (Isopar M, Kraton G 1702, Kraton G 1650, Tinogard)+30% sunflower oil
Prototype Composition 4 (P4): 60% gelled composition (Isopar M, Kraton G 1702, Kraton G 1650, Tinogard)+40% sunflower oil
Prototype Composition 5 (P5): 50% gelled composition (Isopar M, Kraton G 1702, Kraton G 1650, Tinogard)+50% sunflower oil
Prototype Composition 6 (P6): 50% gelled composition (Isopar M, Kraton G 1702, Kraton G 1650, Tinogard)+50% dodecanol

In comparison to the six Prototype Compositions, three marketed, benchmark compositions (B1-B3) were also evaluated. The properties of the three marketed, benchmark compositions were as follows:
Benchmark Composition 1 (B1): Cyclopentasiloxane, Dimethiconol, Fragrance, Sunflower oil, and Coconut oil
Benchmark Composition 2 (B2): Dimethicone, Cyclopentasiloxane, Water, Fragrance, and Sea buckthorn oil
Benchmark Composition 3 (B3): Cyclopentasiloxane, Isododecane, C12-C15 Alkyl Benzoate, Kukui oil, and Phenyl trimethicone

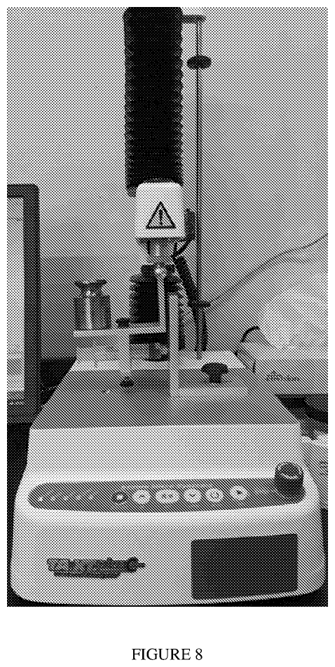

The testing setup for evaluation of lubricity is depicted in FIG. 8. Testing parameters were as follows:

| Test mode | Tension | |
|---|---|---|
| Pre-test speed | 15 | mm/s |
| Post-test speed | 10 | mm/s |
| Target mode | Distance | |
| Distance | 45 | mm |

| | |
|---|---|
| Count | 10 |
| Trigger type | Button |
| Fixture | Custom |

Syringe with 1 ml product & 500 g weight

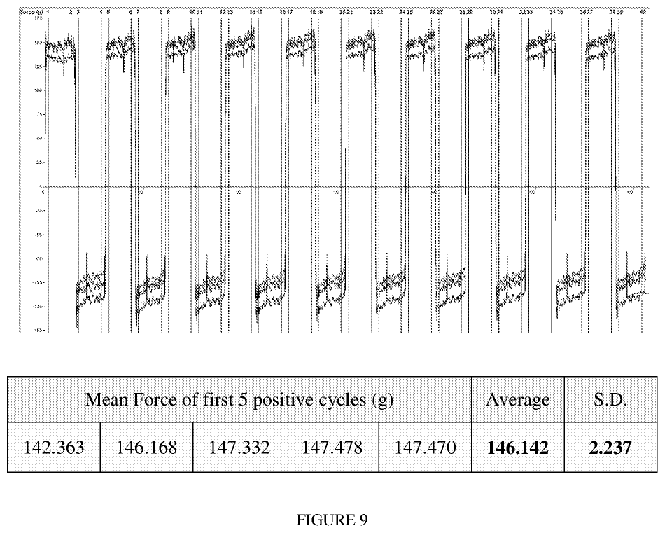

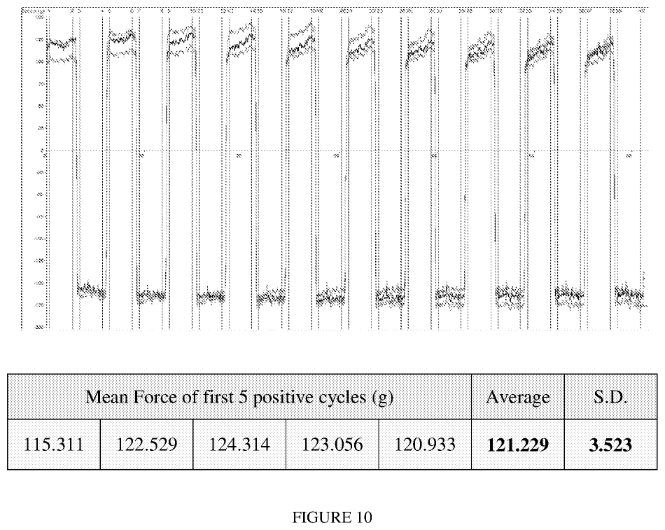

FIG. 9 and FIG. 10 graphically demonstrate the lubricity (friction reduction) of Benchmark Composition B2 (FIG. 9) compared to Prototype Composition P5 (FIG. 10).

### Lubricity

The lubricity (e.g., friction reduction) of the Benchmark Compositions B1-B3 were compared to Prototype Compositions P1-P6. Lubricity was evaluated as the friction is the force of the syringe tip moving against the post which is reduced by a drop of the product.

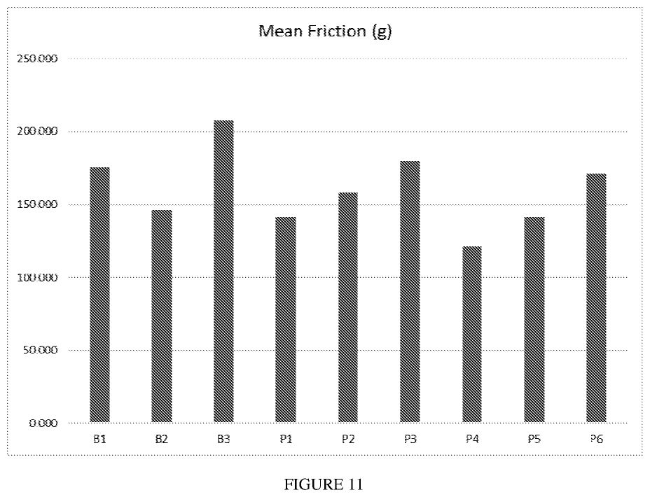

FIG. 11 displays the lubricity (g) of B1-B3 compared to P1-P6. The Benchmark Composition B2 was the most lubricious benchmark composition. In comparison, Prototype Compositions P4 and P5 had the most similar lubricity values to the Benchmark Composition B2.

### EXAMPLE 4

### Refractive Index Evaluation of Gelled Compositions

Refractive Index (RI) properties of gelled compositions of the present disclosure were compared to marketed, benchmark compositions. Refractive Index is an indicative test of formulations or individual products for their ability to impart Shine/Gloss on hair. Refractive Index was analyzed using the Abbemat 200 (Anton Paar Company) according to the standard operating procedure available, for instance, at www.anton-paar.com.

A gelled composition of the present disclosure was combined with an oil or oil-derived component to form a Prototype Composition. In particular, six Prototype Compositions (P1-P6) were formulated as follows using “Gelled Composition 6” as described in Example 1:
Prototype Composition 1 (P1): 90% gelled composition (Isopar M, Kraton G 1702, Kraton G 1650, Tinogard)+10% sunflower oil
Prototype Composition 2 (P2): 80% gelled composition (Isopar M, Kraton G 1702, Kraton G 1650, Tinogard)+20% sunflower oil
Prototype Composition 3 (P3): 70% gelled composition (Isopar M, Kraton G 1702, Kraton G 1650, Tinogard)+30% sunflower oil
Prototype Composition 4 (P4): 60% gelled composition (Isopar M, Kraton G 1702, Kraton G 1650, Tinogard)+40% sunflower oil
Prototype Composition 5 (P5): 50% gelled composition (Isopar M, Kraton G 1702, Kraton G 1650, Tinogard)+50% sunflower oil
Prototype Composition 6 (P6): 50% gelled composition (Isopar M, Kraton G 1702, Kraton G 1650, Tinogard)+50% dodecanol

In comparison to the six Prototype Compositions, three marketed, benchmark compositions (B1-B3) were also evaluated. The properties of the three marketed, benchmark compositions were as follows:
Benchmark Composition 1 (B1): Cyclopentasiloxane, Dimethiconol, Fragrance, Sunflower oil, and Coconut oil
Benchmark Composition 2 (B2): Dimethicone, Cyclopentasiloxane, Water, Fragrance, and Sea buckthorn oil
Benchmark Composition 3 (B3): Cyclopentasiloxane, Isododecane, C12-C15 Alkyl Benzoate, Kukui oil, and Phenyl trimethicone

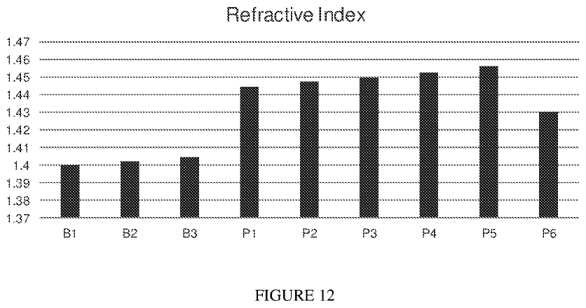

FIG. 12 displays the Refractive Index of B1-B3 compared to P1-P6. The three Benchmark Compositions had similar Refractive Index values. In comparison, all Prototype Compositions had higher RI compared to Benchmark Compositions B1-B3. The highest RI was observed for Prototype Composition P5.

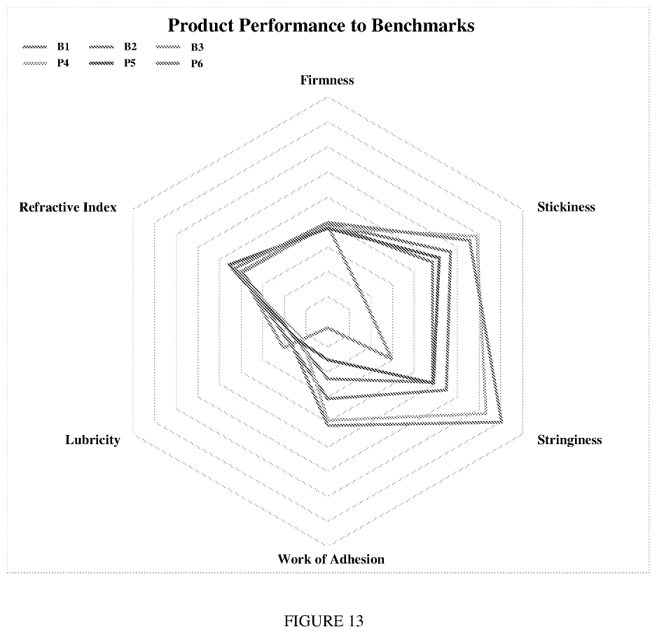

A summary of product performance of various Prototype Compositions compared to the Benchmark Compositions is displayed in FIG. 13. Prototype Compositions P4, P5, and P6 exceed the Benchmark Compositions with respect to Lubricity and Refractive Index properties. Furthermore, Prototype Compositions are comparative to the Benchmark Compositions with respect to Structure (rheology and ease of application), Tackiness, Stringiness, and Work of Adhesion. As a result, the Prototype Compositions allow for versatility in various attributes—shine, rheology, lubricity—based on the unique needs required for formulation.

### EXAMPLE 5

### Rate of Evaporation Evaluation

The instant example compares rate of evaporation of compositions with various solvent systems:
A. Exemplary gelled composition, formulated using “Gelled Composition 6” as described in Example 1
B. Isopar M
C. Cyclopentasiloxane, a 89-90% Cyclomethicone D5 (available as Xiameter PMX-0245 ; https://consumer.dow.com/en-us/pdp.xiameter-pmx-0245-cyclopentasiloxane.01645196z.html?tab=overview&id=01645196z)

The compositions were evaluated as follows. First, an oven was equilibrated to 75° C. (167° F.) and the temperature was maintained. Thereafter, approximately 50 mL of each ingredient was placed in a graduated cylinder. The initial weight (sample+cylinder) was recorded, followed by hourly recordation of weight over 6 hours. Finally, the samples were left in the oven overnight and the weight was measured in the morning.

| Time | A. Gelled | B. Isopar | C. Cyclomethicone |
|---|---|---|---|
| (h) | composition (g) | M (g) | (PMX-0245) |
| Initial | 133 | 128 | 138 |
| 1 | 133 | 128 | 138 |
| 2 | 133 | 128 | 138 |
| 3 | 133 | 128 | 138 |
| 4 | 133 | 128 | 138 |
| 5 | 133 | 128 | 138 |
| 6 | 133 | 128 | 138 |
| Overnight | 133 | 128 | 138 |

There was no observed difference in rate of evaporation expected for the various compositions at physiologically relevant temperatures. However, the evaluation may not be sensitive enough to differentiate the rates of evaporation of the solvents. A more sensitive test involving both heating and rapid air movement across the surface using a fan is contemplated.

1. A gelled composition comprising i) an aliphatic solvent, ii) one or more copolymers, and iii) an antioxidant.

2. The gelled composition of claim 1, wherein the aliphatic solvent is present in the gelled composition at a concentration of about 80% to about 99.9%.

3. The gelled composition of claim 1, wherein the aliphatic solvent is an isoparaffin.

4. The gelled composition of claim 3, wherein the isoparaffin is present in the gelled composition at a concentration of about 80% to about 99.9%.

5. The gelled composition of claim 1, wherein the aliphatic solvent is selected from the group consisting of a C7-8 isoparaffin, a C8-9 isoparaffin, a C10-11 isoparaffin, a C11-12 isoparaffin, a C11-13 isoparaffin, a C13-14 isoparaffin, and any combination thereof.

6. The gelled composition of claim 1, wherein the aliphatic solvent is an Isopar solvent or a compositional equivalent.

7. The gelled composition of claim 1, wherein the aliphatic solvent is an Isopar solvent.

8. The gelled composition of claim 7, wherein the Isopar solvent is selected from the group consisting of Isopar M, Isopar C, Isopar E, Isopar G, Isopar H, Isopar L, Isopar V, and any combination thereof.

9. The gelled composition of claim 7, wherein the Isopar solvent is Isopar M.

10. The gelled composition of claim 9, wherein the Isopar M is present in the gelled composition at a concentration of about 80% to about 99.9%.

11. (canceled)

12. (canceled)

13. (canceled)

14. (canceled)

15. The gelled composition of claim 1, wherein the gelled composition comprises a first copolymer, wherein the first copolymer is Kraton G 1702, and optionally comprises a second copolymer.

16. (canceled)

17. (canceled)

18. (canceled)

19. The gelled composition of claim 1, wherein the gelled composition comprises a first copolymer, wherein the first copolymer is Kraton G 1650, and optionally comprises a second copolymer.

20. (canceled)

21. The gelled composition of claim 1, wherein the gelled composition comprises two copolymers, wherein one copolymer is Kraton G 1702 and the second copolymer is Kraton G 1650.

22. (canceled)

23. (canceled)

24. (canceled)

25. (canceled)

26. The gelled composition of claim 1, wherein the gelled composition is substantially free of a silicone.

27. The gelled composition of claim 1, wherein the gelled composition does not comprise a silicone.

28. (canceled)

29. (canceled)

30. (canceled)

31. The gelled composition of claim 1, wherein the gelled composition has a viscosity from about 5,000 cps to about 10,000 cps.

32. The gelled composition of claim 1, wherein the gelled composition has a viscosity from about 10,000 cps to about 20,000 cps.

33. A composition comprising the gelled composition of claim 1 and an oil.

34. The composition of claim 33, wherein the oil is sunflower oil.

35. The composition of claim 33, wherein the oil is selected from the group consisting of apricot oil, avocado oil, borage oil, castor oil, coconut oil, evening primrose oil, gold of pleasure oil, grape seed oil, hazelnut oil, jojoba oil, macadamia oil, passionflower oil, peach kernel oil, rice bran oil, safflower oil, sesame oil, shea oleine oil, sweet almond oil, almond oil, wheat germ oil, sunflower oil, and any combination thereof.

36. (canceled)

37. (canceled)

38. (canceled)
